(19) United States
(12) Patent Application Publication
(10) Pub. No.: US 2005/0192225 A1
(43) Pub. Date: Sep. 1, 2005

(54) Methods for treating Parkinson's disease

(76) Inventor: Richard Lloyd Bowen, Raleigh, NC (US)

Correspondence Address: COVINGTON & BURLING;ATTN: PATENT DOCKETING, 1201 PENNSYLVANIA AVENUE, N.W., WASHINGTON, DC, 20004-2401, US

(21) Appl. No.: 11/053,445

(22) Filed: Feb. 9, 2005

Related U.S. Application Data
(63) Continuation-in-part of application No. 10/321,579, filed on Dec. 18, 2002.

(51) Int. Cl. A61K 38/17

(52) U.S. Cl. 514/12

(57) ABSTRACT

Methods for treating Parkinson's disease include administration of agents that increase or regulate blood or tissue levels, production, function, or activity of inhibins or follistatin, or that decrease or regulate blood or tissue levels, production, function, or activity of activins.

This application claims priority under 35 U.S.C. §120 to U.S. application Ser. No. 10/321,579, filed Dec. 18, 2002. The entirety of U.S. application Ser. No. 10/321,579 is hereby incorporated by reference herein.

### FIELD OF THE INVENTION

This invention relates to methods for treating Parkinson's disease. More particularly, this invention relates to the administration of agents that increase or regulate blood or tissue levels, production, function, or activity of inhibins or follistatin, or that decrease or regulate blood or tissue levels, production, function, or activity of activins, thus preventing or delaying the onset of and the progression of Parkinson's disease.

### BACKGROUND

Parkinson's disease is an age-related neurodegenerative disease with a mean age at onset of 55 years. There are approximately 1 million people with the disease in the United States. Ninety-five percent of cases are sporadic and have no apparent genetic linkage. Parkinson's disease causes significant morbidity and increased mortality among sufferers. Costs associated with disability, lost productivity, and pharmaceutical treatment for Parkinson's disease patients are more than $26 billion dollars per year.

Parkinson's disease is characterized by resting tremor, bradykinesia, hypokinesia, akinesia, rigidity, stooped posture, instability, and in twenty-five percent or more of patients, cognitive abnormalities manifested as passivity, delayed responsiveness, depression, and dementia (Dauer, W. and Przedborski, S. Parkinson's disease: mechanisms and models. Neuron 39:889-909 (2003)). The neuropathological characteristics of Parkinson's disease are the loss of dopaminergic neurons in the substantia nigra pars compacta, the presence of intraneuronal proteinaceous inclusions known as Lewy bodies, and a reduction in striatal dopamine levels (Schapira, A. H. V. and Olanow, C. W. Neuroprotection in Parkinson disease. Mysteries, myths and misconceptions. Journal of the American Medical Association 291:358-364 (2004)).

In Parkinson's disease, more neurons are lost from the ventrolateral and caudal portions of the substantia nigra pars compacta, compared to normal aging during which neurons of the dorsomedial aspect are affected (Fearnley, J. M. and Lees, A. J. Ageing and Parkinson's disease: substantia nigra regional selectivity. Brain 114:2283-2301 (1991)). The striatal dopaminergic nerve terminals appear to be the primary structures that degenerate prior to neuronal cell body destruction (Bernheimer, H., Birkmayer, W., Hornykiewicz, O., Jellinger, K. and Seitelberger, F. Brain dopamine and the syndromes of Parkinson and Huntington. Clinical, morphological and neurochemical correlations. Journal of Neurological Science 20:415-455 (1973)).

Current Treatments of Parkinson's Disease

Currently available therapies for Parkinson's disease are symptomatic therapies, and no curative or disease-modifying therapy is known.

Levodopa treatment is the mainstay therapy for management of the disease, but long-term treatment is associated with development of motor fluctuations and dyskinesia within 5 years (Rascol, O., Brooks, D. J., Korczyn, A. D., DeDeyn, P. P., Clarke, C. E., Lang, A. E. A five-year study of the incidence of dyskinesia in patients with early Parkinson's disease who were treated with ropinirole or levodopa. New England Journal of Medicine 342:1484-1491 (2000)). Anticholinergic drugs, which inhibit cholinergic neurons whose actions oppose dopamine, are used to treat tremors and rigidity. Catechol-O-methyltransferase inhibitors prevent the peripheral and central metabolism of levodopa to 3-O-methyidopa, thus prolonging the “wearing-off” time of levodopa.

Inhibitors of monoamine oxidase-B (the enzyme that catalyzes dopamine) prolong the action of dopamine in the brain and have been shown to provide symptomatic benefits, but such inhibitors are not known to have neuroprotective effects. Drugs in the monoamine oxidase-B inhibitor class include selegiline and amantadine (Romrell, J., Fernandez, H. H., Okun, M. S. Rationale for current therapies in Parkinson's disease. Expert Opinions in Pharmacotherapeutics 4:1747-1761 (2003)).

While there have been multiple neuroprotective trials performed for Parkinson's disease, the data are conflicting and inconclusive. Neuroprotection studies have examined the effects of antioxidants, including vitamin E and coenzyme Q, and the dopamine agonist pramipexole (Schapira, A. H. V. and Olanow, C. W. Neuroprotection in Parkinson disease. Mysteries, myths and misconceptions. Journal of the American Medical Association 291:358-364 (2004)).

Activins and Inhibins

Activins and inhibins are dimeric proteins composed of non-covalently linked subunits: α subunit and/or β subunits A, B, C, D, and E (Fang et al., 1996; Hotten et al., 1996; Oda et al., 1995; Vale et al., 1990). The α-subunit is expressed primarily in reproductive tissues and is directly correlated to oogenesis and spermatogenesis, while β-subunits are expressed in reproductive and numerous other tissues (Hubner et al., 1999). Inhibin A is composed of an a subunit and a βA subunit. Inhibin B consists of an α subunit and a βB subunit (Bernard et al., 2001). Activin A is composed of two βA subunits, activin AB is composed of one βA subunit and one βB subunit, and activin B is composed of two βB subunits (Halvorson and DeCherney, 1996). Since β-subunits C, D, and E have only recently been identified, little is known about their interactions with the other subunits (Hotten et al., 1996; Mellor et al., 2000; O'Bryan et al., 2000).

Since activins were found to stimulate gonadotropin secretion, they were initially identified as members of the hypothalamic-pituitary-gonadal axis. (Ling et al., 1986; Vale et al., 1986). Stimulation of gonadotropin production by activins is inhibited by inhibins and follistatin. Inhibin binds to and inactivates activin receptors in a competitive manner. This inhibitory action is significantly enhanced in tissues whose cell membranes express betaglycan. Follistatin irreversibly binds to activins and prevents them from binding to activin receptors (DeKretser et al., 2002; Gray et al., 2002). Activins have since been found to be members of the transforming growth factor beta (TGF-β) family of proteins and to be involved in many non-reproductive functions.

Activins and their receptors are ubiquitously expressed and have important functions in the regulation of cellular differentiation and apoptosis (Baer, H., Friess, H., Abou-Shady, M., Berberat, P., Zimmermann, A., Gold, L., Korc, M., and Buchler, M. Transforming growth factor betas and their receptors in human liver cirrhosis. Eur J Gastroenterol Hepatol 10, 1031-1039 (1998). Baldwin, R. L., Friess, H., Yokoyama, M., Lopez, M. E., Kobrin, M. S., Buchler, M. W., and Korc, M. Attenuated ALK5 receptor expression in human pancreatic cancer: correlation with resistance to growth inhibition. Int J Cancer 67, 283-288 (1996). Dewulf, N., Verschueren, K., Lonnoy, O., Moren, A., Grimsby, S., VandeSpiegle, K., Miyazono, K., Huylebroeck, D., and TenDijke, P. Distinct spatial and temporal expression patterns of two type I receptors for bone morphogenetic proteins during mouse embryogenesis. Endocrinology 136, 2652-2663 (1995). Kitten, A. M., Kreisberg, J. I., and Olson, M. S. Expression of osteogenic protein-1 mRNA in cultured kidney cells. J Cell Physiol 181, 410-415 (1999). Li, G., Borger, M. A., Williams, W. G., Weisel, R. D., Mickle, D. A., Wigle, E. D., and Li, R. K. Regional overexpression of insulin-like growth factor-I and transforming growth factor-betal in the myocardium of patients with hypertrophic obstructive cardiomyopathy. J Thorac Cardiovasc Surg 123, 89-95 (2002). Schluns, K. S., Grutkoski, P. S., Cook, J. E., Engelmann, G. L., and Le, P. T. Human thymic epithelial cells produce TGF-beta 3 and express TGF-beta receptors. Int Immunol 7, 1681-1690 (1995)). Follistatin, which has wide tissue expression, likely functions to regulate both the reproductive and non-reproductive actions of activins in an autocrine/paracrine fashion.

The manner by which activins affect cellular function is complex (Nishimura et al., 1998). Activins bind to a type II serine threonine kinase receptor, ActRII or ActRIIB, to form a complex that recruits and activates an activin type I receptor ALK4, leading to activation of downstream signaling through Smad proteins (reviewed in Gray, P. C., Bilezikjian, L. M., Vale, W. W. Antagonism of activin by inhibin and inhibin receptors: a functional role for betaglycan. Molecular and Cellular Endocrinology 188:254-260 (2002)). Smads then participate directly in the regulation of gene expression by binding to DNA, interacting with transcription factors, and recruiting corepressors or coactivators to specific promoters (van Grunsven et al., 2002). Inhibin also binds activin type II receptors, and inhibin and activin share a binding site on ActRII.

It remains to be determined if there are unique inhibin receptors, but inhibin has been shown to bind to ActRII (Zimmerman and Mathews, 2001). Inhibins appear to function primarily to regulate the activity of activins by binding the activin receptor and interfering with the ability of activin to activate its receptor (Bernard et al., 2001). Even further complexity is evidenced by how the receptor affinity of inhibins is greatly influenced by the presence or absence of betaglycan content of the cell membrane. Betaglycan has been reported to facilitate binding of inhibin to the activin receptor ActRII to form a complex that recruits ALK4. The association of ActRII with inhibin and betaglycan prevents activin from binding to the receptor and leads to blockage of activin signals (Gray, P. C., Bilezikjian, L. M., Vale, W. W. Antagonism of activin by inhibin and inhibin receptors: a functional role for betaglycan. Molecular and Cellular Endocrinology 188:254-260 (2002)).

### SUMMARY OF THE INVENTION

In accordance with the present invention, an increase in the blood or tissue levels, production, function, or activity of various inhibins and/or follistatin or a decrease in the blood or tissue levels, production, function, or activity of activins prevents or delays the death of dopaminergic neurons in the brain, particularly in the substantia nigra pars compacta, which is the hallmark of Parkinson's disease. Increased blood or tissue levels, production, function, or activity of inhibin or follistatin or decreased blood or tissue levels, production, function, or activity of activin is expected to block or delay pathogenic changes that cause neuronal death, including oxidative stress, mitochondrial dysfunction, excitotoxicity, and inflammation.

### DETAILED DESCRIPTION

In an embodiment of the invention, the blood or tissue levels, production, function, or activity of inhibin isotypes or follistatin are increased to levels that are as high as possible without causing significant adverse side effects. In another embodiment, the blood or tissue levels, production, function, or activity of activin isotypes are decreased to levels that are as low as possible without causing significant adverse side effects.

According to another embodiment of the invention, inhibin, follistatin, or analogues of either of these are used to increase the blood or tissue levels, production, function, or activity of inhibin or follistatin. Agents or interventions that increase blood or tissue levels, production, function, or activity of inhibin or follistatin include but are not limited to recombinant or natural forms of these hormones, agents that stimulate production of these hormones, gene therapeutics that increase production of these hormones, passive immunization against inhibitors of these hormones, ribonucleic acid interference to prevent expression of proteins that inhibit these hormones, dominant negative expression of genes to prevent inhibition of these hormones, and agents or interventions that increase cell membrane betaglycan content.

Agents or interventions that decrease blood or tissue levels, production, function, or activity of activins include but are not limited to vaccines that stimulate the production of antibodies that block the activity of activin or its receptor or receptors of other proteins that would stimulate the activity of activins, and any analogues or salts of the foregoing agents. Agents that decrease blood or tissue levels, production, function, or activity of activins include but are not limited to inhibin or follistatin, gene therapeutics that decrease production of activin, passive immunization against activin, ribonucleic acid interference to prevent the expression of activins or activin receptors, dominant negative expression of genes that stimulate the expression of or activity of activins or activin receptors, and any analogues or salts of the foregoing agents.

Administration of other agents, including agents not yet known, that increase or regulate blood or tissue levels, production, function, or activity of inhibins or follistatin, or that decrease or regulate blood or tissue levels, production, function, or activity of activin or activin receptors, are encompassed by the present invention.

In another embodiment of the invention, the administration of agents that decrease the expression of smad proteins that are known to be activated by activins, or that increase the expression of smad proteins that are known to be inhibited by activins, is expected to decrease or regulate blood or tissue levels, production, function, or activity of activins.

According to a further embodiment of the invention, the administration of agents such as other TGF-β proteins that are known to inhibit smads that activins stimulate or that stimulate smads that activins inhibit is expected to decrease or regulate blood or tissue levels, production, function, or activity of activins.

1. A method for preventing or delaying neuronal death in the brain of a patient, comprising:
administering to the patient a therapeutically effective amount of an agent that increases blood or tissue levels, production, function, or activity of at least one of inhibin and follistatin within the patient.

2-20. (canceled)
